(19) United States
(12) Patent Application Publication
(10) Pub. No.: US 2024/0245747 A1
(43) Pub. Date: Jul. 25, 2024

(54) METHOD FOR IMPROVING COGNITIVE FUNCTION AND A DIETARY SUPPLEMENT FOR USE IN THE METHOD

(71) Applicant: RB Health (US) LLC, Parsippany, NJ (US)

(72) Inventors: Lauren Roukia BRINK, Parsippany, NJ (US); Neeraj PANDEY, Slough (GB); Jane Mary BARRACATO, Parsippany, NJ (US)

(21) Appl. No.: 18/417,113

(22) Filed: Jan. 19, 2024

Related U.S. Application Data
(60) Provisional application No. 63/480,711, filed on Jan. 20, 2023.

(51) Int. Cl. A61K 36/9062; A61K 31/4415; A61K 31/519; A61K 31/685; A61K 31/714; A61K 36/74

(52) U.S. Cl. CPC A61K 36/9062; A61K 31/4415; A61K 31/519; A61K 31/685; A61K 31/714; A61K 36/74

(57) ABSTRACT

The present disclosure relates generally to a method of promoting cognitive function in a target subject by the administration of a dietary supplement composition. Additionally, the disclosure relates to a dietary supplement composition which promotes cognitive function. In particular, the dietary supplement composition comprises phosphatidylserine, coffee fruit extract and Alpinia galanga extract.

### CROSS-REFERENCE TO RELATED APPLICATIONS/INCORPORATION BY REFERENCE STATEMENT

The subject application claims benefit under 35 USC § 119(e) of U.S. Provisional Application No. 63/480,711, filed Jan. 20, 2023. The entire contents of the above-referenced patent application are hereby expressly incorporated herein by reference.

### TECHNICAL FIELD

The present disclosure relates generally to a method of promoting cognitive function in a target subject by the administration of a dietary supplement composition. Additionally, the disclosure relates to a dietary supplement composition which promotes cognitive function. In particular, the dietary supplement composition comprises phosphatidylserine, coffee fruit extract, and Alpinia galanga extract.

### BACKGROUND

A dietary supplement is a product which is designed to be consumed by a human or animal to supplement the diet to provide nutrients in order to increase the intake of one or more nutrients. Often, such supplements contain vitamins and minerals which have been found to be essential to life. However, dietary supplements may also contain ingredients which have a beneficial biological effect, but which are not necessarily deemed essential to life.

Phosphatidylserine is a type of phospholipid found in cell membranes and particularly in the cells of the brain. It is vital for cognitive function as it is key to supporting cell function in the brain where it impacts the brain's structure and signal system. The intake of phosphatidylserine from food alone has declined over recent years. Studies have revealed phosphatidylserine contributes to cognitive function in individuals of various age groups. It is believed that young people can synthesize enough phosphatidylserine to maintain healthy brain function. However, during aging the phosphatidylserine content in the brain diminishes leading to neuronal chemical changes. Dietary supplements for cognitive impairment published in Acts Esp Psiquiatr 2017; 45 (Suppl.1):37-47 states that “cerebral aging is associated with changes in the lipid composition of neuronal membranes” so it has been suggested that the treatment with phospholipids, like phosphatidylcholine and phosphatidylserine, could favour cognitive improvement.

Coffee fruit is the skin and rind which surrounds a coffee bean. It has been proven to elevate the body's level of the neuroprotein brain-derived neurotrophic factor (BDNF). BDNF is a vital neuroprotein that is known to strengthen connections between brain cells and helps establish new connections. It is also important for neuronal survival and growth, serves as a neurotransmitter modulator and participates in neuronal plasticity which is essential for learning and memory. It has been demonstrated in the British Journal of Nutrition, Vol. 110, Issue 3 that the consumption of whole coffee fruit can support the production of BDNF in a human.

Alpinia galanga (L.) Sw. (Zingiberaceae), is a plant of the ginger family, and is also known by various other names, including great galangal, lengkuas blue ginger, Thai ginger, and Siamese ginger. Alpinia galanga is a native to Southeast Asia, where the rhizome is used as an herb and spice in cookery and traditional medicine.

Alpinia galanga has been shown in human clinical trials to enhance alertness and focus.

The foregoing discussion is presented solely to provide a better understanding of the nature of the problems confronting the art and should not be construed in any way as an admission to prior art.

The present disclosure is directed, in an embodiment, to provide a method for improving brain function. In another embodiment, it is directed to a dietary supplement composition which improves brain function.

### DETAILED DESCRIPTION

Reference will be made in detail to the embodiments of the present disclosure, one or more examples of which are set forth hereinbelow. Each example is provided by way of explanation of the dietary supplement composition of the present disclosure and is not a limitation. In fact, it will be apparent to those skilled in the art that various modifications and variations can be made to the teachings of the present disclosure without departing from the scope of the disclosure. For instance, features, illustrated or described as part of one embodiment can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

### Definitions

As used herein, all terms are intended to have their ordinary meaning in the art unless specifically defined.

“Adult” means a subject in age from 18 years upwards.

“Modulate” or “modulating” means exerting a modifying, controlling and/or regulating influence. In some embodiments, the term “modulating” means exhibiting an increasing or stimulatory effect on the level/amount of a particular component. In other embodiments, “modulating” means exhibiting a decreasing or inhibitory effect on the level/amount of a particular component.

All percentages, parts and ratios as used herein are by weight of the total formulation, unless otherwise specified.

The present disclosure relates generally to a method for improving brain function via administration of a dietary supplement composition comprising phosphatidylserine, coffee fruit extract and Alpinia galanga extract. Additionally, the disclosure relates to a dietary supplement composition comprising phosphatidylserine coffee fruit extract and Alpinia galanga extract which is suitable for administration to a human subject.

The dietary supplement composition of the present disclosure may be substantially free of any optional or selected ingredients described herein, provided that the remaining nutritional composition still contains all of the required ingredients or features described herein.

All references to singular characteristics or limitations of the present disclosure shall include the corresponding plural characteristic or limitation, and vice versa, unless otherwise specified or clearly implied to the contrary by the context in which the reference is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint (s) of any range. Any reference to a range should be considered as providing support for any subset within that range.

According to a first non-limiting aspect of the present disclosure, there is provided a method of improving cognitive function in a target subject, the method comprising the step of administering a dietary supplement composition comprising phosphatidylserine, coffee fruit extract, and Alpinia galanga extract.

According to a second non-limiting aspect of the present disclosure, there is provided a dietary supplement composition comprising phosphatidylserine, coffee fruit extract, and Alpinia galanga extract.

According to a third non-limiting aspect of the present disclosure, there is provided a dosage regimen for administering a dietary supplement composition comprising phosphatidylserine, coffee fruit extract, and Alpinia galanga extract to a target subject wherein the regimen comprises a daily dosage amount of phosphatidylserine, coffee fruit extract, and Alpinia galanga extract which may be delivered in one or more unit doses.

In accordance with the foregoing objectives and others, the present disclosure provides a method for promoting cognitive function in a target subject. In certain embodiments, the target subject is a mammal including humans and companion animals. In some embodiments, the target subject is an adult human. In another non-limiting embodiment, the present disclosure is directed to a composition suitable for use as a dietary supplement by combining phosphatidylserine, coffee fruit extract and Alpinia galanga extract in a single composition. In some embodiments, it has surprisingly been found that a dietary supplement composition comprising coffee fruit extract, phosphatidylserine and Alpinia galanga extract has a beneficial effect which is greater than the effect expected from phosphatidylserine and coffee fruit extract when administered individually or in combination.

The present disclosure is directed to a method of improving brain function by the administration of a dietary supplement composition comprising phosphatidylserine coffee fruit extract, and Alpinia galanga extract and to the composition itself. The administration of the dietary supplement composition results in improved focus, memory, learning, accuracy concentration, reasoning and mental alertness or a combination thereof in a target subject. In certain embodiments, the combination of phosphatidylserine coffee fruit extract and Alpinia galanga extract is believed to have additive and/or synergistic beneficial effect over the expected effect from the administration of phosphatidylserine or coffee fruit extract or Alpinia galanga extract alone or a combination of two of the three ingredients. In some embodiments, as a result of the improved brain function other aspects such as a target subject's behavior may be modulated.

The method of improving brain function in a target subject is achieved by the administration of a dietary supplement composition comprising phosphatidylserine coffee fruit extract and Alpinia galanga extract via the gut-brain axis. Thus, in some embodiments, the present disclosure is directed to a method of modulating gut-brain communication, the method comprising administering to a target subject a nutritional composition comprising phosphatidylserine coffee fruit extract and Alpinia galanga extract.

### Phosphatidylserine

In one non-limiting embodiment, the method comprises administering a dietary supplement composition comprising phosphatidylserine. In one non-limiting embodiment, phosphatidylserine may be sourced from a plant. In particular, phosphatidylserine may be sourced from soybeans. This is a preferred (but non-limiting) alternative to animal sources to render the dietary supplement composition vegetarian or vegan.

There is no recommended daily intake for phosphatidylserine, however, the inventors have found suitable quantities for achieving the effects of improved brain function. In some non-limiting embodiments, the composition of the present disclosure may comprise phosphatidylserine in a unit dose from about 40 mg to about 400 mg. In some non-limiting embodiments, the composition of the present disclosure may comprise phosphatidylserine in a unit dose from about 75 mg to about 400 mg, or from about 90 mg to about 300 mg, or from about 50 mg to about 150 mg, or about 100 mg.

In certain particular (but non-limiting) embodiments, the composition comprises about 100 mg phosphatidylserine.

In a non-limiting embodiment, a dietary supplement composition may be administered to a target subject one or more times a day. In this case, a number of unit doses can be administered to achieve a desired daily intake amount of phosphatidylserine, for example from about 40 mg to about 400 mg or it can be administered in a single dose.

### Coffee Fruit Extract

In one embodiment, the method comprises administering a dietary supplement composition comprising coffee fruit extract.

In an embodiment, a source of coffee fruit extract is from the whole fruit of the Coffea arabica plant. The extract may optionally be treated to remove caffeine.

There is no recommended daily intact for coffee fruit extract, however, the inventors have found suitable quantities for achieving the effects of improved brain function. In some non-limiting embodiments, the composition of the present disclosure may comprise coffee fruit extract in a unit dose from about 80 mg to about 300 mg per serving. In some non-limiting embodiments, the composition of the present disclosure may comprise coffee fruit extract in a unit dose from about 100 mg to about 240 mg, or about 200 mg.

In a particular (but non-limiting) embodiment, the composition comprises about 200 mg coffee fruit extract.

In a non-limiting embodiment, a dietary supplement composition may be administered to a target subject one or more times a day. In this case, a number of unit doses can be provided to achieve a desired daily intake of coffee fruit extract, for example from about 80 mg to about 300 mg or it can be administered in a single unit dose.

Alpinia galanga

In one embodiment, the method comprises administering a dietary supplement composition comprising an Alpinia galanga extract.

In an embodiment the Alpinia galanga extract is a water-soluble extract that is substantially free of methyl eugenol. Advantageously the absence of methyl eugenol diminishes the caffeine “crash” that is often follows consumption of a caffeinated composition.

“Substantially” herein means that the composition comprises less than about 10%, such as less than about 5%, less than about 1%, or less than about 0.1% of methyl eugenol.

There is no recommended daily intact for Alpinia galanga extract, however, the inventors have found suitable quantities for achieving the effects of improved brain function. In some non-limiting embodiments, the composition of the present disclosure may comprise from about 100 to about 900 mg of Alpinia galanga extract, or from about 200 mg to about 800 mg, or from about 300 mg to about 700 mg, or from about 400 mg to about 600 mg, or from about 450 mg to about 550 mg, or about 300 mg.

In a particular (but non-limiting) embodiment, the composition comprises about 300 mg of Alpinia galanga extract.

In a non-limiting embodiment, a dietary supplement composition may be administered to a target subject one or more times a day. In this case, a number of unit doses can be administered to achieve a desired daily intake amount of Alpinia galanga extract, for example from about 100 mg to about 900 mg or it can be administered in a single dose. In some non-limiting embodiments, the composition of the present disclosure may be administered in one or more unit doses to deliver Alpinia galanga extract in an amount from about 200 mg to about 800 mg, or from about 300 mg to about 700 mg, or from about 400 mg to about 600 mg, or from about 450 mg to about 550 mg.

The extract of Alpinia galanga that is substantially free of methyl eugenol may also include a ratio of polyphenols to polysaccharides of about 1:1.6 to about 1:12.

It is hypothesised that the polyphenol, polysaccharide and pyrocatecollic type tannins found in a water-soluble extract of Alpinia galanga may be particularly responsible for the cognitive benefit in a subject. Specifically, a water-extraction method results in an extract having ratios and amounts of these compounds that provide superior improvements in cognitive performance over and above those provided by a natural powder or from an alcoholic extraction.

In certain non-limiting embodiments, a water-soluble extract of Alpinia galanga comprises not less than about 3% total polyphenols, not less than about 4% flavonoids, not less than 20% polysaccharides and not less than 1% pyrocatecollic type tannins.

Together the phosphatidylserine, coffee fruit extract and Alpinia galanga support neuronal health and increase BDNF in a target subject. Though not wishing to be bound by theory, it is believed that phosphatidylserine, coffee fruit extract and Alpinia galanga extract interact synergistically with one another to provide unexpected benefits when compared to the effect achieved by each of these ingredients taken alone. Improved brain function can be demonstrated in a target subject by an improvement in performance in at least one of accuracy, concentration, focus, learning, memory reasoning and alertness. Examples of how this can be verified is through the performance of tasks, for example by assessments, puzzles, games and mental exercises which can be monitored by recording the results and the time taken to complete each task. This can be tracked and monitored to determine the improvement in brain function from before and during the administration of the dietary supplement composition.

In particular, alertness is defined as achieving or maintaining a state of high sensitivity to incoming stimuli. The Attention network test (ANT) may be employed to demonstrate increased alertness in a human subject. The ANT test provides a behavioural measure of the efficiency of different components of attention networks separately within a single task.

The dietary supplement composition of the present disclosure may comprise additional ingredients. Non-limiting examples of such ingredients can include but are not limited to further active ingredients, excipients, flow agents, processing aids, solvents, carriers, antioxidants, lubricants, colorants, flavourings and the like and will be apparent to those skilled in the art in view of the disclosure herein.

The nutritional composition may optionally include, but is not limited to, one or more of the following vitamins or derivations thereof: vitamin B6, vitamin B12 and/or folate. The source of vitamin B6 may be pyridoxine, pyridoxal, pyridoxamine, or pyridoxine hydrochloride and be in an amount in a range of from about 1 mg to about 4 mg, such as (but not limited to) a range of from about 1.5 mg to about 2.5 mg per unit dose. The source of vitamin B12 may be cobalamin, methylcobalamin, deoxyadenosylcobalamin, cyanocobalamin, hydroxocobalamin, or adenosylcobalamin and be in an amount in a range of from about 1 μg to about 11 μg, such as (but not limited to) a range of from about 2 μg to about 8 μg or a range of from about 2.4 μg to about 4.8 μg per unit dose. The source of folate may be from folic acid, folacin, or pteroylglutamic acid. In certain non-limiting embodiments, the folate is provided by folic acid in an amount in a range of from about 0.2 mg to about 1.0 mg, such as (but not limited to) a range of from about 0.4 mg to about 0.8 mg per unit dose. When the combination of vitamin B6, vitamin B12 and folate are included together in the dietary supplement composition the improved brain function can be further demonstrated by improved reasoning capability of the target subject. Again, this can be proven through the performance of tasks.

In other embodiments, excipients that can be used include but are not limited to rice bran, microcrystalline cellulose, gelatin, pectin, hydroxypropyl methylcellulose, hydroxypropyl cellulose, polyvinylpyrrolidone, calcium phosphate, dicalcium phosphate, sucrose, starch, maltodextrin, inulin, agar, acacia, dextrose and stearic acid. If rice bran is used in the dietary supplement composition, then it is present in an amount of about 4 mg to about 25 mg per unit dose.

In other embodiments, the comprises at least one flow agent. Flow agents can include but are not limited to silicon dioxide, talc, magnesium silicate, calcium silicate, calcium phosphate and starch. In an embodiment, the total amount of flow agents may be from about 4 mg to about 15 mg per unit dose.

In other embodiments, the dietary supplement composition comprises other active ingredients. For example, one or more vitamins and/or minerals may also be added in to the nutritional composition in amounts sufficient to supply at least some of the daily nutritional requirements of a subject. It is to be understood by one of ordinary skill in the art that vitamin and mineral requirements will vary, for example, based on the age of the target individual. Thus, the embodiments are not intended to limit the nutritional composition to a particular age group but, rather, to provide a range of acceptable vitamin and mineral components.

In other embodiments, the dietary supplement composition comprises at least one lubricant. Lubricants can include but are not limited to magnesium stearate, calcium stearate, glycerine monostearate, glyceryl behenate, hydrogenated vegetable oil, mineral oil, medium chain triglycerides, sodium lauryl sulfate, stearic acid and talc.

In other embodiments, the dietary supplement composition comprises at least one colorant. Colorant can include coloring agents derived either from natural or synthetically prepared sources. Any coloring agents approved by the Food and Drug Administration for use in human food and/or drugs are contemplated by the present disclosure.

In other embodiments, the dietary supplement composition comprises flavoring. Flavoring can include but is not limited to ethyl acetate, ethyl vanillin, fumaric acid, leucine, malic acid, maltol, menthol, vanillin, acesulfame potassium, citric acid, fructose, ethylcellulose, tartaric acid, trehalose and xylitol.

The compositions according to the present disclosure may be formulated in a variety of forms for administration and comprise an effective amount of phosphatidylserine, coffee fruit extract and Alpinia galanga, by which is meant an amount sufficient to provide health benefits to a target subject. In some embodiments, the effective amounts of phosphatidylserine and coffee fruit extract are sufficient to improve brain function.

The compositions presently disclosed may be administered orally. Any dosage forms, and combinations thereof, are contemplated by the present disclosure. Examples of dosage forms suitable for oral delivery include, without limitation, chewable tablets, elixirs, liquids, solutions, suspensions, emulsions, capsules, soft capsules, hard capsules, tablet, powder, gummy, melt in the mouth, caplets, lozenges, confections and combinations thereof. The capsules may be hard capsules, e.g. two-piece capsules, or soft capsules, e.g. softgels. In at least one embodiment, the capsule is a hard shell.

The composition may be encapsulated in a capsule made from a non-mammalian source such as hydroxypropyl methyl cellulose, carrageenan and pectin which renders the dietary supplement composition vegetarian or vegan. Such hardshell capsules can be prepared, by way of example, without limitation, by dispersing the formulation in an appropriate vehicle, such as rice bran, and then encapsulating in a film to form a capsule in any machinery known to those in the industry of manufacturing.

### EXAMPLES

Examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the examples.

### Examples 1-4

Dietary supplement compositions of the disclosure may comprise the ingredients in the following amounts to form a unit dose.

TABLE 1

| Ingredients | Example 1 | Example 2 | Example 3 | Example 4 |
|---|---|---|---|---|
| Phosphatidylserine (mg) | 40 | 100 | 200 | 300 |
| Coffee fruit extract (mg) | 80 | 200 | 400 | 600 |
| Alpinia galanga extract (mg) | 120 | 300 | 600 | 900 |
| Rice bran (mg) | 7 | 18 | 21 | 24 |
| Microcrystalline cellulose (mg) | 100 | 130 | 120 | 150 |
| Silicon dioxide (mg) | 6 | 7 | 8 | 8 |
| Pyridoxine hydrochloride (vitamin B6) (mg) | | 2 | | 4 |
| Cyanocobalamin (vitamin B12) (mcg) | | 2.4 | | 8 |
| Folic acid (from folate) (mcg) | | 680 | | 1000 |
| Magnesium stearate (mg) | | 3 | | 4 |

The dietary supplement composition of Example 1 may be suitable for use in a dosage regime where three unit doses can be taken in a day to provide 120 mg of phosphatidylserine, 240 mg of coffee fruit extract and 360 mg of Alpinia galanga. The dietary supplement composition of Example 2 may be suitable for use in a dosage regimen where one or more unit doses are administered per day.

The dietary supplement compositions of Examples 3 and 4 may be suitable for use in a dosage regimen where one unit dose is administered per day.

### Example 5

One exemplary composition comprising phosphatidylserine coffee fruit extract and Alpinia galanga extract according to the present disclosure was prepared according to the formulation provided in Table 2. Phosphatidylserine was purchased from Enzymotec under the brand name Sharp-PS® which contains further ingredients. Coffee fruit extract was purchased from Futureceuticals. Alpinia galanga was purchased from Omniactives Inc under the brand name EnExtra®.

TABLE 2

| Ingredient | Amount per unit dose | |
|---|---|---|
| Phosphatidylserine | 100 | mg |
| Coffee fruit extract | 200 | mg |
| Alpinia galanga extract | 300 | mg |
| Rice bran | 8 | mg |
| Microcrystalline cellulose | 132.3 | mg |
| Silicon dioxide | 8 | mg |

The composition was encapsulated in a film comprising: hydroxypropyl methylcellulose sold by Suheung to form a hardshell capsule. The resulting unit dose had a total fill of 468.3 mg with a shell weighing 95 mg. Such a unit dose may be used in a dosage regimen where one unit dose is administered per day.

### Example 6

Another exemplary composition comprising phosphatidylserine, coffee fruit extract and Alpinia galanga extract according to the present disclosure is provided in Table 3. Phosphatidylserine was purchased from Enzymotec under the brand name Sharp-PS® which contains further ingredients. Coffee fruit extract was purchased from Futureceuticals. Alpinia galanga was purchased from Omniactives Inc under the brand name EnExtra®.

TABLE 3

| Ingredients | Amount per unit dose | |
|---|---|---|
| Phosphatidylserine | 100 | mg |
| Coffee fruit extract | 200 | mg |
| Alpinia galanga | 300 | mg |
| Pyridoxine hydrochloride (vitamin B6) | 1.70 | mg |
| Cyanocobalamin (vitamin B12) | 0.024 | mg |
| Folic acid (from folate) | 0.4 | mg |
| Rice bran | 21 | mg |
| Microcrystalline cellulose | 118 | mg |
| Silicon dioxide | 8 | mg |
| Magnesium stearate | 4 | mg |

The composition was encapsulated in a film comprising: hydroxypropyl methylcellulose sold by Suheung to form a hardshell capsule. The resulting unit dose soft gel had a total fill of 595.03 mg with a shell weighing 118 mg. Such a unit dose may be used in a dosage regimen where one unit dose is administered per day.

The compositions made according to the present disclosure provide benefits to a target subject when administered. These benefits include improved brain function demonstrated by improved focus, memory, learning, accuracy, concentration, reasoning and mental alertness.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references. Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

### Non-Limiting Illustrative Embodiments

Illustrative embodiment 1. A method of improving cognitive function in a target subject, the method comprising the step of administering a dietary supplement composition to the target subject, wherein the dietary supplement comprises phosphatidylserine, coffee fruit extract, and Alpinia galanga extract.

Illustrative embodiment 2. The method according to illustrative embodiment 1, wherein the phosphatidylserine is present in the dietary supplement in an amount in a range of from about 75 mg to about 400 mg, such as (but not limited to) a range of from about 90 mg to about 300 mg, or a range of from about 50 to about 150 mg, or an amount of about 100 mg.

Illustrative embodiment 3. The method according to illustrative embodiment 1 or 2, wherein the coffee fruit extract is present in the dietary supplement in an amount in a range of from about 80 mg to about 300 mg, such as (but not limited to) a range of from about 100 mg to about 240 mg, or an amount of about 200 mg.

Illustrative embodiment 3A. The method according to any of illustrative embodiments 1-3, wherein the coffee fruit extract has been decaffeinated.

Illustrative embodiment 4. The method according to any of illustrative embodiments 1-3A, wherein the Alpinia galanga extract is present in the dietary supplement in an amount in a range of from about 100 to about 900 mg, such as (but not limited to) a range of from about 200 mg to about 800 mg, a range of from about 300 mg to about 700 mg, a range of from about 400 mg to about 600 mg, a range of from about 450 mg to about 550 mg, or an amount of about 300 mg.

Illustrative embodiment 5. The method according to any of illustrative embodiments 1-4, wherein the Alpinia galanga extract is a water-soluble extract and is substantially free of methyl eugenol.

Illustrative embodiment 6. The method according to any of illustrative embodiments 1-5, wherein the supplement further comprises one or more vitamins.

Illustrative embodiment 7. The method according to illustrative embodiment 6, wherein the vitamins comprise vitamin B6.

Illustrative embodiment 7A. The method according to illustrative embodiment 7, wherein the vitamin B6 is provided by one or more of pyridoxine, pyridoxal, pyridoxamine, and pyridoxine hydrochloride.

Illustrative embodiment 7B. The method according to illustrative embodiment 7 or 7A, wherein the vitamin B6 is present in the dietary supplement in an amount in a range of from about 1 mg to about 4 mg, such as (but not limited to) a range of from about 1.5 mg to about 2.5 mg.

Illustrative embodiment 8. The method according to any of illustrative embodiments 6-7B, wherein the vitamins comprise vitamin B12.

Illustrative embodiment 8A. The method according to illustrative embodiment 8, wherein the vitamin B12 is provided by one or more of cobalamin, methylcobalamin, deoxyadenosylcobalamin, cyanocobalamin, hydroxocobalamin, and adenosylcobalamin.

Illustrative embodiment 8B. The method according to illustrative embodiment 8 or 8A, wherein the vitamin B12 is present in the dietary supplement in an amount in a range of from about 1 μg to about 11 μg, such as (but not limited to) a range of from about 2 μg to about 8 μg, or a range of from about 2.4 μg to about 4.8 μg.

Illustrative embodiment 9. The method according to any of illustrative embodiments 1-8B, wherein the dietary supplement further comprises folate.

Illustrative embodiment 9A. The method according to illustrative embodiment 9, wherein the folate is provided by at least one of folic acid, folacin, and pteroylglutamic acid.

Illustrative embodiment 9B. The method according to illustrative embodiment 9 or 9A, wherein the folate is present in the dietary supplement in an amount in a range of from about 0.2 mg to about 1.0 mg, such as (but not limited to) a range of from about 0.4 mg to about 0.8 mg.

Illustrative embodiment 10. The method according to any of illustrative embodiments 1-9, wherein the dietary supplement is administered in a form selected from the group consisting of solution, capsule, tablet, gummy, chewable, melt in the mouth, liquid, or caplet.

Illustrative embodiment 11. The method according to any of illustrative embodiments 1-10, wherein the target subject is an adult human.

Illustrative embodiment 12. A dietary supplement composition comprising: phosphatidylserine, coffee fruit extract, and Alpinia galanga extract.

Illustrative embodiment 13. The dietary supplement composition according to illustrative embodiment 12, wherein the phosphatidylserine is present in an amount in a range of from about 75 mg to about 400 mg, such as (but not limited to) a range of from about 90 mg to about 300 mg, or a range of from about 50 to about 150 mg, or an amount of about 100 mg.

Illustrative embodiment 14. The dietary supplement composition according to illustrative embodiment 12 or 13, wherein the coffee fruit extract is present in an amount in a range of from about 80 mg to about 300 mg, such as a range of from about 100 mg to about 240 mg, or an amount of about 200 mg.

Illustrative embodiment 14A. The dietary supplement composition according to any of illustrative embodiments 12-14, wherein the coffee fruit extract has been decaffeinated.

Illustrative embodiment 15. The dietary supplement composition according to any of illustrative embodiments 12-14A, wherein the Alpinia galanga extract is present in an amount in a range of from about 100 to about 900 mg, such as (but not limited to) a range of from about 200 mg to about 800 mg, a range of from about 300 mg to about 700 mg, a range of from about 400 mg to about 600 mg, a range of from about 450 mg to about 550 mg, or an amount of about 300 mg.

Illustrative embodiment 16. The dietary supplement composition according to any of illustrative embodiments 12-15, wherein the Alpinia galanga extract is a water-soluble extract and is substantially free of methyl eugenol.

Illustrative embodiment 17. The dietary supplement composition according any of illustrative embodiments 12-16, wherein the supplement further comprises one or more vitamins.

Illustrative embodiment 18. The dietary supplement composition according to illustrative embodiment 17, further comprising vitamin B6.

Illustrative embodiment 18A. The dietary supplement composition according to illustrative embodiment 18, wherein vitamin B6 is provided by one or more of pyridoxine, pyridoxal, pyridoxamine, and pyridoxine hydrochloride.

Illustrative embodiment 18B. The dietary supplement composition according to illustrative embodiment 18 or 18A, wherein the vitamin B6 is present in an amount in a range of from about 1 mg to about 4 mg, such as (but not limited to) a range of from about 1.5 mg to about 2.5 mg.

Illustrative embodiment 19. The dietary supplement composition according to any of illustrative embodiments 17-18B, further comprising vitamin B12.

Illustrative embodiment 19A. The dietary supplement composition according to illustrative embodiment 19, wherein the vitamin B12 is provided by one or more of cobalamin, methylcobalamin, deoxyadenosylcobalamin, cyanocobalamin, hydroxocobalamin, and adenosylcobalamin.

Illustrative embodiment 19B. The dietary supplement composition according to illustrative embodiment 19 or 19A, wherein the vitamin B12 is present in an amount in a range of from about 1 μg to about 11 μg, such as (but not limited to) a range of from about 2 μg to about 8 μg, or a range of from about 2.4 μg to about 4.8 μg.

Illustrative embodiment 20. The dietary supplement composition according to any of illustrative embodiments 12-19B, further comprising folate.

Illustrative embodiment 20A. The dietary supplement composition according to illustrative embodiment 20, wherein the folate is provided by at least one of folic acid, folacin, and pteroylglutamic acid.

Illustrative embodiment 20B. The dietary supplement composition according to illustrative embodiment 20 or 20A, wherein folic acid is present in an amount in a range of from about 0.2 to about 1.0 mg, such as (but not limited to) a range of from about 0.4 mg to about 0.8 mg.

Illustrative embodiment 21. The dietary supplement composition according to any of illustrative embodiments 12-20B, wherein the supplement is in a form selected from the group consisting of solution, capsule, tablet, gummy, chewable, melt in the mouth, liquid, or caplet.

Illustrative embodiment 22. A dosage regimen for administering a dietary supplement composition comprising phosphatidylserine, coffee fruit extract, and Alpinia galanga extract to a target subject, wherein the regimen comprises a daily dosage amount of phosphatidylserine, coffee fruit extract, and Alpinia galanga extract which may be delivered in one or more unit doses.

1. A method of improving cognitive function in a target subject, the method comprising the step of:
administering a dietary supplement composition to the target subject, wherein the dietary supplement composition comprises phosphatidylserine, coffee fruit extract, and Alpinia galanga extract.

2. The method according to claim 1, wherein the phosphatidylserine is present in the dietary supplement composition in an amount in a range of from about 75 mg to about 400 mg.

3. The method according to claim 1, wherein the coffee fruit extract is present in the dietary supplement composition in an amount in a range of from about 80 mg to about 300 mg.

4. The method according to claim 1, wherein the Alpinia galanga extract is present in the dietary supplement composition in an amount in a range of from about 100 mg to about 900 mg.

5. The method according to claim 1, wherein the Alpinia galanga extract is a water-soluble extract and is substantially free of methyl eugenol.

6. The method according to claim 1, wherein the dietary supplement composition further comprises one or more vitamins.

7. The method according to claim 6, wherein the vitamins comprise vitamin B6.

8. The method according to claim 6, wherein the vitamins comprise vitamin B12.

9. The method according to claim 1, wherein the dietary supplement composition further comprises folate.

10. The method according to claim 1, wherein the dietary supplement composition is administered in a form selected from the group consisting of solution, capsule, tablet, gummy, chewable, melt in the mouth, liquid, or caplet.

11. The method according to claim 1, wherein the target subject is an adult human.

12. A dietary supplement composition, comprising:
phosphatidylserine;
coffee fruit extract; and
Alpinia galanga extract.

13. The dietary supplement composition according to claim 12, wherein the phosphatidylserine is present in an amount in a range of from about 75 mg to about 400 mg.

14. The dietary supplement composition according to claim 13, wherein the coffee fruit extract is present in an amount in a range of from about 80 mg to about 300 mg.

15. The dietary supplement composition according to 12, wherein the Alpinia galanga extract is present in an amount in a range of from about 100 to about 900 mg.

16. The dietary supplement composition according to claim 12, wherein the Alpinia galanga extract is a water-soluble extract and is substantially free of methyl eugenol.

17. The dietary supplement composition according any of claim 12, wherein the supplement further comprises one or more vitamins.

18. The dietary supplement composition according to claim 17, further comprising vitamin B6.

19. The dietary supplement composition according to claim 17, further comprising vitamin B12.

20. The dietary supplement composition according to claim 12, further comprising folate.

21. The dietary supplement composition according to claim 12, wherein the supplement is in a form selected from the group consisting of solution, capsule, tablet, gummy, chewable, melt in the mouth, liquid, or caplet.

22. A dosage regimen for administering a dietary supplement composition comprising phosphatidylserine, coffee fruit extract, and Alpinia galanga extract to a target subject wherein the regimen comprises a daily dosage amount of phosphatidylserine, coffee fruit extract, and Alpinia galanga extract which may be delivered in one or more unit doses.
